(19) United States
(12) Patent Application Publication
(10) Pub. No.: US 2009/0012620 A1
(43) Pub. Date: Jan. 8, 2009

(54) Implantable Cervical Fusion Device

(76) Inventors: Jim Youssef, Durango, CO (US); Daniel Riew, St. Louis, MO (US); Jeffrey Wang, Sherman Oaks, CA (US); Jj Abitbol, San Diego, CA (US); Pierce Dalton Nunley, Grand Cane, LA (US)

Correspondence Address: PATENTMETRIX, 14252 CULVER DR. BOX 914, IRVINE, CA, 92604, US

(21) Appl. No.: 11/774,006

(22) Filed: Jul. 6, 2007

(51) Int. Cl. A61F 2/44

(52) U.S. Cl. 623/17.16

(57) ABSTRACT

The present invention relates to an implantable intervertebral device that is made of a bone body substantially conforming in size and shape with the enplates of adjacent vertebrae. The bone body has a top surface, a bottom surface, and, a cavity, which extends from the top surface to the bottom surface. The cavity is filled with osteoconductive or osteoinductive graft material.

### FIELD OF THE INVENTION

The present invention relates generally to an implantable intervertebral fusion device and, more specifically, to allograft bone devices with an anatomical shape that effectively conforms to, and adheres to, the endplates of the adjacent vertebras. The present invention is also related to improved cervical allograft bone devices with an anatomical shape that promotes better healing and natural lordosis of the cervical spine.

### BACKGROUND

The vertebral column is a bio-mechanical arrangement composed largely of ligaments, muscles, vertebrae, and intervertebral discs. The bio-mechanical functions of the spine include: (1) support of the body, which involves the transfer of the weight and the bending movements of the head, trunk and arms to the pelvis and legs; (2) complex physiological motion between these parts; and (3) protection of the spinal cord and nerve roots.

When viewed laterally, the vertebral column has three main regions that represent the curvature of the column: cervical region, thoracic region and lumbar region. The first seven vertebrae form the cervical spinal region. The next twelve vertebrae form the thoracic spinal region or mid-back. The lower portion of the spine (i.e. lumbar spinal region) is usually made of five vertebrae. As described in greater detail below, lordosis describes an inward curvature of a portion of the vertebral column. The cervical and lumbar regions of the vertebral column are normally lordotic, that is, they are set in a curve that has its convexity in front and concavity behind, in the context of human anatomy. Thus, the cervical spinal region curves slightly inward, the thoracic slightly outward, and the lumbar slightly inward. Even though the lower portion of the spine holds most of the body's weight, each region relies upon the strength of the other regions for proper functioning.

As populations age, it is anticipated that there will be an increase in adverse spinal conditions which are characteristic of aging. For example, with aging comes an increase in the degeneration of the intervertebral disc. Disabling mechanical pain resulting from disc degeneration is often treated surgically with an interbody fusion.

The primary purpose of the intervertebral discs, located between the endplates of the adjacent vertebrae, is to distribute forces between vertebrae, stabilize the spine, and cushion vertebral bodies. Thus the intervertebral disc acts as a shock absorber for the spinal column. A normal intervertebral disc includes a semi-gelatinous component which is surrounded by an outer ring called the annulus fibrosus. In a healthy spine, the annulus fibrosus prevents the gelatinous component from protruding outside the disc space.

Spinal discs may be displaced or damaged as a result of disease, trauma, aging or injury to the spine, and more specifically, injury to the cervical region. While the cervical region is very flexible, it is at a high risk due to injury from strong sudden movements, such as whiplash-type injuries. This high risk of trauma is due to the limited muscle support that exists in the cervical region and the amount of weight it has to support. For example, an average head weighs around 15 pounds, which is a lot of weight for small, thin set of bones and soft tissues to bear. In some cases, sudden, strong head movements can also cause damage.

Generally, the only relief from the disability caused by degenerated spinal discs is a discectomy, or surgical removal of the intervertebral disc followed by fusions of portions of the adjacent vertebrae. The removal of the damaged or unhealthy disc without reconstruction would allow the disc space to collapse, resulting in further instability of the spine, abnormal joint mechanics, premature development of arthritis or nerve damage, in addition to severe pain. To prevent the intervertebral space from collapsing, a structure must be placed within the intervertebral space to provide support.

For example, in early spinal fusion techniques, bone material, or bone osteogenic fusion devices were simply placed between the transverse processes of adjacent vertebrae. The osteogenic fusion material consisted of cortical-cancellous bone which was not strong enough to support the weight of the spinal column at the instrumented level. Consequently, the spine was stabilized by way of a plate or a rod spanning the affected vertebrae.

For example, U.S. Pat. No. 4,604,995, assigned to Stephens, David C. and Morgan, Craig D., discloses “a surgical implant for imparting stability to the thoraco-lumbar spine by fixation of the implant to the spine with segmental spinal instrumentation, the implant comprising: a unitary rod having a generally rectangular configuration formed by a pair of spaced apart branches substantially mirror image duplicates of one another and substantially equally spaced apart along their entire length; a bight end piece interconnecting the branch pair at one end portion thereof; and a gate forming end piece connected to close the other end portion of the branch pair except for a small gate opening to provide access to the space between the branch pair.”

With this technique, once the fusion occurs, the hardware maintaining the stability of the spine becomes superfluous. There are other several disadvantages associated with the use of the abovementioned metal implants. Solid body metal implants do not effectively enable bone in-growth which may lead to the eventual failure of the implant. Surface porosity in such solid implants does not correct this problem because it will not allow sufficient in-growth to provide a solid bone mass strong enough to withstand the loads of the spine. Attention was then turned to implants, or interbody fusion devices, which could be interposed between the adjacent vertebrae, maintain the stability of the disc interspace, and still permit fusion or arthrodesis.

For example, U.S. Pat. No. 4,961,740, assigned to Centerpulse USA Inc., discloses “a fusion cage adapted for promoting fusion of one or more bone structures when bone-growth-inducing substance is packed into the fusion cage, comprising: a cage body defining a cavity with an inner surface; said cavity adapted to be packed with the bone-growth-inducing substance; said cage body defining an outer surface; means for defining threads on the outer surface of the cage body and adapted for biting into the bone structure; said threads defining means including a plurality of threads which define valleys there between; a plurality of perforations provided in the valleys of the threads for providing communication between the outer surface and the cavity in order to allow immediate contact between the one or more bone structures and the bone-growth-inducing substance packed into the fusion cage”.

U.S. Pat. No. 5,026,373, assigned to Surgical Dynamics, discloses “a method for surgically preparing two adjacent bony structures for implanting a hollow cylindrical fusion cage that has an external, substantially continuous helical thread which defines a plurality of turns with a valley between adjacent turns and that is perforated in the valley between adjacent turns of the thread, said method comprising the steps of: (a) drilling a pilot hole laterally between said bony structures, (b) inserting a pilot rod into the pilot hole, (c) fitting a hollow drill over the pilot rod, (d) with the hollow drill, enlarging said pilot hole to form a bore that penetrates into the cortical bone of each of said bony structures, and (e) tapping a female thread into the wall of said bore, the crown of which female thread penetrates into the cancellous portion of each of said bony structures, which female thread can mate with the helical thread of the fusion cage.”

The abovementioned intervertebral fusion device has substantial disadvantages, however. The metallic supporting frame of the prior art fusion cages is not osteoconductive and therefore does not form a strong mechanical attachment to a patient's bone tissue. This can lead to graft necrosis, poor fusion and poor stability. Moreover, many of these devices are difficult to machine and therefore expensive. Furthermore, the fusion cages may stress shield the bone graft, increasing the time required for fusion to occur. The abovementioned implants further requires a special tool and additional preparation of the adjacent vertebral bodies to ensure fusion.

In addition, the use of bone graft materials in the prior art metal cage fusion devices presents several disadvantages. Autografts, bone material surgically removed from the patient, are undesirable because the donor site may not yield a sufficient quantity of graft material. The additional surgery to extract the autograft also increases the risk of infection, persistent pain, and may reduce structural integrity at the donor site.

U.S. Pat. No. 5,489,308 assigned to Zimmer Spine, Inc., discloses “an implant for insertion into a bore formed between opposing vertebrae of a spine where said vertebrae are separated by a spacing with a disk material having an annulus disposed within said spacing, said implant comprising: a rigid body having a leading end and a trailing end spaced apart by a longitudinal axis of said body; said body comprising at least exposed threads disposed at least partially between said leading end and said trailing end; said threads selected to engage vertebra material and draw said body along a direction of said axis upon rotation of said body about said axis; said body having a hollow, generally cylindrical shell with said threads disposed on an exterior surface of said shell; said body having means defining a chamber disposed within said body and said body is provided with a rib disposed within said cylindrical shell and extending radially inwardly toward said longitudinal axis, said rib dividing said chamber into a leading end chamber and a trailing end chamber, and said rib including at least a rigid extension extending between and connecting diametrically opposed sides of said body; said body having means defining at least one opening formed through said body in communication with said chamber and with said opening extending generally radially to said axis; and said body having a transverse dimension generally transverse to said longitudinal axis and dimensioned so as to be greater than said bore for said body to urge said opposing vertebrae apart and to stretch said annulus upon insertion of said body into said bore between said vertebrae with a portion of said body opposing a first of said opposing vertebrae and with an opposite side of said body opposing a second of said opposing vertebrae.”

One problem with the implant devices of the type mentioned above is that they tend not to maintain the normal curvature of the spine. In a healthy state, the cervical and lumbar areas of the human spine curve convexly forward. Normal lordosis results, at least in significant measure, from the normal wedge-shaped nature of the spaces between adjacent pairs of the cervical and lumbar vertebrae, and the normal wedge-shaped nature of the intervertebral discs that fill these spaces. Loss of lordosis and proper intervertebral spacing may result in an increased risk of degeneration to other intervertebral discs located adjacent to the fusion level due to the alteration of the overall mechanics of the spine.

A further problem with the abovementioned implant is that the cylindrical geometry of the engaging element tends to provide a small area of contact between the engaging element and the vertebrae. The small engaging surface tends to contribute to subsidence or deformation of the cortical layer of the vertebrae adjacent to the engaging element. Moreover, the small engaging surface provides less contact between the bone graft material encased in the device and the adjacent vertebrae. Exposure of the bone graft material to the surface of the vertebrae is important because the greater the area of contact, the greater the possibility of having a successful fusion.

U.S. Pat. No. 6,143,033 discloses “an allogenic intervertebral implant for use when surgical fusion of vertebral bodies is indicated. The implant comprises an annular plug conforming in size and shape with the end plates of adjacent vertebrae and has a plurality of teeth positioned on the top and bottom surfaces for interlocking with the adjacent vertebrae. The teeth preferably have a pyramid shape or a saw-tooth shape.” The teeth 105 of a prior art implant are shown in FIG. 1.

U.S. Pat. No. 6,986,788 discloses “an allogenic intervertebral implant for use when surgical fusion of vertebral bodies is indicated. The implant comprises a piece of allogenic bone conforming in size and shape with a portion of an end plate of the vertebrae and has a wedge-shaped profile with a plurality of teeth located on top and bottom surfaces.” The teeth 205 of the implant 200 have a pyramidal shape, as shown in FIG. 2.

However, the above-mentioned implants are not sufficiently effective at preventing expulsion of the implant. The surfaces of the implants, whether saw tooth 105 or pyramidal 205, do not effectively provide implant stability.

In addition, there are special considerations in conducting anterior or posterior cervical fusion procedures. Specifically, the movements of flexion and extension of the head take place predominantly at the joint between the first cervical vertebra and the occipital bone, the atlanto-occipital joint. However, the cervical spine is comparatively mobile, and some component of this movement is due to flexion and extension of the vertebral column itself.

The movement of rotating the head to left and right happens almost entirely at the joint between the first and second cervical vertebrae, the atlanto-axial joint. A small amount of rotation of the vertebral column itself contributes to the movement. The movement of lateral flexion of the neck, as when the subject attempts to place the ear against the tip of the shoulder, happens due to the movement in the vertebral column itself.

The general characteristics of the third through sixth cervical vertebrae are described below. It should be noted that the first, second, and seventh vertebrae have extraordinary characteristics independent of the third through sixth. The body of these four vertebrae is smaller and broader from side to side than from front to back.

The anterior and posterior surfaces are flattened and of equal depth; the former is places on a lower level that the latter, and its inferior border is prolonged downward, so as to overlap the upper and forepart of the vertebra below.

The upper surface is concave transversely, and presents a projecting lip on either side; the lower surface is concave from front to back, convex from side to side, and presents laterally shallow concavities which receive the corresponding projecting lips of the underlying vertebra.

The pedicles are directed laterally and backward, and are attached to the body midway between its upper and lower borders, so that the superior vertebral notch is as deep as the inferior, but it is, at the same time, narrower.

The laminae are narrow, and thinner above than below, the vertebral foramen is large, and of a triangular form. The spoinous process is short and bifid, the two divisions being often of unequal size.

The superior and inferior articular processes of neighboring vertebra often fuse on either or both sides to form an articular pillar, a column of bone which projects laterally from the junction of the pedicle and lamina. The articular facets are flat and of an oval form:
the superior face backward, upward, and slightly medially
the inferior face forward, downward, and slightly laterally.

The transverse processes are each pierced by the foramen transversarium, which, in the upper six vertebrae gives passage to the vertebral artery and vein, as well as a plexus of sympathetic nerves. Each process consists of an anterior and a posterior part. The anterior portion is the homologue of the rib in the thoracic region, and is therefore names the costal process or costal element. It arises from the side of the body, is directed laterally in front of the foramen, and ends in a tubercle, the anterior tubercle. The posterior part, the true transverse process, springs from the vertebral arch behind the foramen, and is directed forward and laterally; it ends in a flattened vertical tubercle, the posterior tubercle.

The two parts are joined, outside the foramen, by a bar of bone which exhibits a deep sulcus on its upper surface for the passage of corresponding spinal nerve.

Anterior spinal fusion surgeries are commonly done in conjunction with an anterior cervical discectomy. For many patients, cervical spine fusion surgery is often done to eliminate motion at a vertebral segment. Decreasing the motion at a painful motion segment should decrease the pain at that segment. Achieving the fusion also serves to maintain adequate space for the decompressed spinal cord and/or nerve roots. The fusion may also prevent the spine from falling into a collapsed deformity (kyphosis).

In the light of the abovementioned disadvantages, there is a need for improved methods and systems that can provide effective, efficient and fast intervertebral fusion. Specifically, an implantable, intervertebral cervical fusion device is needed that conforms to the endplates of the patient's adjacent vertebrae, maintains the normal disc spacing, and normal curvature or lordosis of the cervical spine. Further, an approach is needed that maximizes the probability of success of bone fusion, provides instant stability to the cervical spine while fusion occurs, is easily implantable, and minimizes trauma to the patient and the possibility of surgical and post-surgical complications.

### SUMMARY OF THE INVENTION

The present invention relates to an implantable intervertebral fusion device for use when surgical fusion of vertebral bodies is indicated.

In one embodiment, the invention is an implantable intervertebral device, comprising a bone body substantially conforming in size and shape with the endplates of adjacent vertebrae wherein, the bone body comprises a top surface and a bottom surface and a through cavity.

In one embodiment, the invention is an implantable intervertebral device, comprising a bone body substantially conforming in size and shape with the endplates of adjacent vertebrae wherein, the bone body comprises a top surface and a bottom surface and a through cavity having a cancellous center for better end-to-end growth and fusion.

Optionally, the bone body is cut and machined into annular shapes.

Optionally, the bone body has lateral corners which are rounded or chamfered.

Optionally, in another embodiment, the bone body further comprises a top surface and a bottom surface.

Optionally, the top and bottom surfaces are flat planar surfaces.

Optionally, the top and bottom surfaces comprises a macro-structure having plurality of footings and grooves that define a space, said space being covered by a micro-structure. Still optionally, the micro-structure mimics cancellous bone architecture and/or has osteoconductive or osteoinductive qualities. The device is made of bone which is cut and machined into annular shapes.

Optionally, the through cavity is filled with porous osteoconductive or osteoinductive graft material to promote bony end to end growth and fusion.

Optionally, the through cavity is filled with a high precision cancellous center.

### BRIEF DESCRIPTION OF THE DRAWINGS

These and other features and advantages of the present invention will be appreciated, as they become better understood by reference to the following Detailed Description when considered in connection with the accompanying drawings, wherein:

FIG. 1 is a side view of the teeth of a prior art implant;

FIG. 2 is a perspective view of a prior art implant with pyramidal teeth;

FIG. 3 is a three-dimensional top view of one embodiment of a cervical intervertebral fusion device of the present invention;

FIG. 4 is a two-dimensional top view of one embodiment of a cervical intervertebral fusion device of the present invention, shown in FIG. 3;

FIG. 5 is a two-dimensional side view of one embodiment of a cervical intervertebral fusion device of the present invention, shown in FIG. 3;

FIG. 6 is a schematic diagram of an anterior cervical fusion (ACF) surgery illustrating the implantation of the cervical intervertebral fusion device of the present invention, shown in FIG. 3;

FIG. 7 is a three-dimensional top view of another embodiment of a cervical intervertebral fusion device of the present invention;

FIG. 8 is a two-dimensional top view of the another embodiment of the cervical intervertebral fusion device of the present invention, shown in FIG. 7;

FIG. 9 is a two-dimensional side view of the another embodiment of the cervical intervertebral fusion device of the present invention, shown in FIG. 7;

FIG. 10A is a two-dimensional side view of one embodiment of a cervical intervertebral fusion device of the present invention, having a cancellous center; and

FIG. 10B is a two-dimensional view of one embodiment of a cervical intervertebral fusion device of the present invention, having a cancellous center.

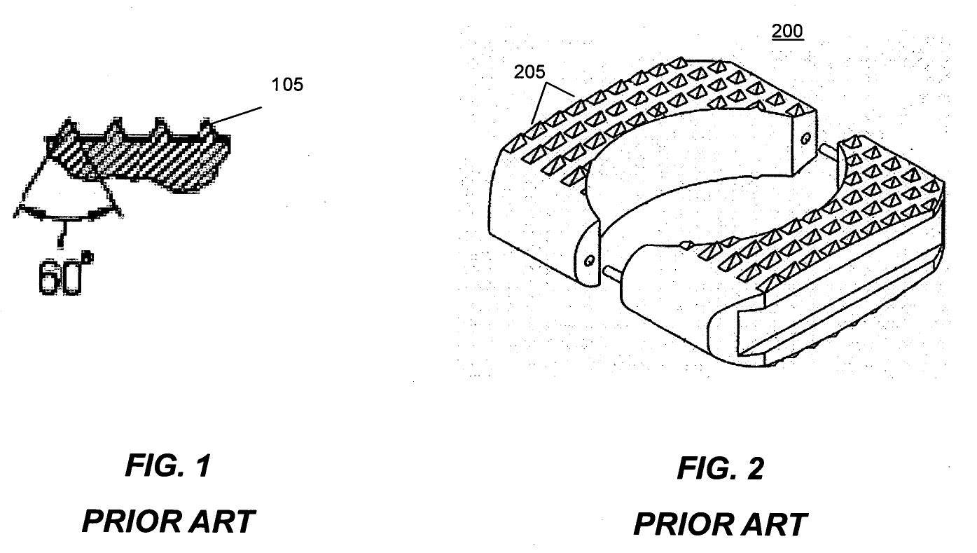

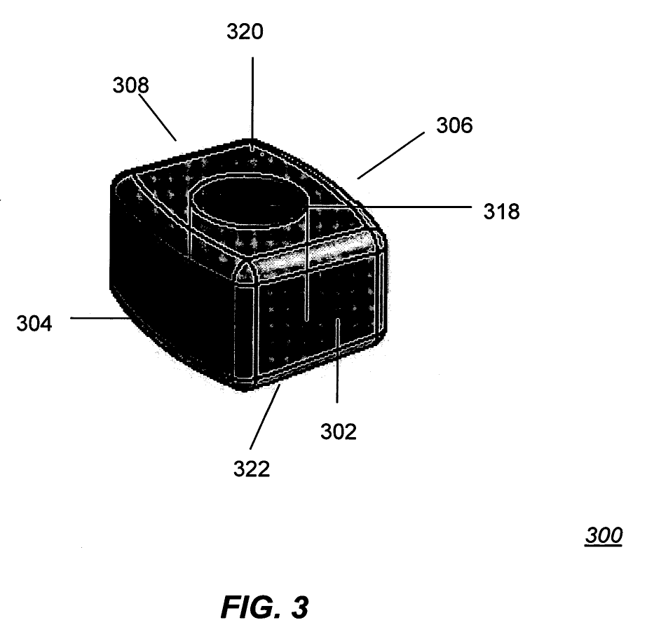

### DETAILED DESCRIPTION

The present invention relates to an implantable intervertebral fusion device for use when surgical fusion of vertebral bodies is indicated. In one embodiment, the invention is an implantable intervertebral device, comprising a bone body substantially conforming in size and shape with the endplates of adjacent vertebrae wherein. In one embodiment, the bone body is cut and machined into annular shapes.

In one embodiment, the bone body has lateral corners which are rounded or chamfered. In addition, to facilitate the insertion of implant and to adapt anatomically to the curvature of the vertebrae endplates, the lateral corners of the device are rounded or chamfered. The rounded or chamfered edges enable the intervertebral fusion device to slide between the endplates while minimizing the necessary distraction of the endplates. In another embodiment, resorbable and/or nonresorbable fixation devices, such as screws, could be placed on the endplates in front of the implant to improve initial fixation.

In one embodiment, the bone body further comprises a top surface and a bottom surface.

In one embodiment, the top and bottom surfaces are flat planar surfaces.

In one embodiment, the top and bottom surfaces comprise a macro-structure having plurality of footings and grooves that define a space, said space being covered by a micro-structure.

In one embodiment, the micro-structure mimics cancellous bone architecture and/or has osteoconductive or osteoinductive qualities.

In one embodiment, although the intervertebral fusion device is a solid piece of allogenic cortical bone, the device can be provided with a hollow interior or through cavity to form an interior space. In one embodiment, this interior space can be filled with bone chips, any other osteoconductive surfacing or surface treatment, or any osteoinductive or other bone growth stimulation coating material to further promote the formation of new bone.

In one embodiment, the hollow interior or through cavity is packed with porous osteoconductive or osteoinductive graft material to promote bony end to end growth and fusion.

In one embodiment, the hollow interior or through cavity is filled with a high precision milled cancellous center to promote bony end to end growth and fusion.

The dimensions of the intervertebral implant can be varied to accommodate a patient's anatomy. However, the intervertebral fusion device of the present invention is preferably wide enough to support adjacent vertebrae and is of sufficient height to separate the adjacent vertebrae.

Reference will now be made to specific embodiments of the present invention. The present invention is directed toward multiple embodiments. Language used in this specification should not be interpreted as a general disavowal of any one specific embodiment or used to limit the claims beyond the meaning of the terms used therein.

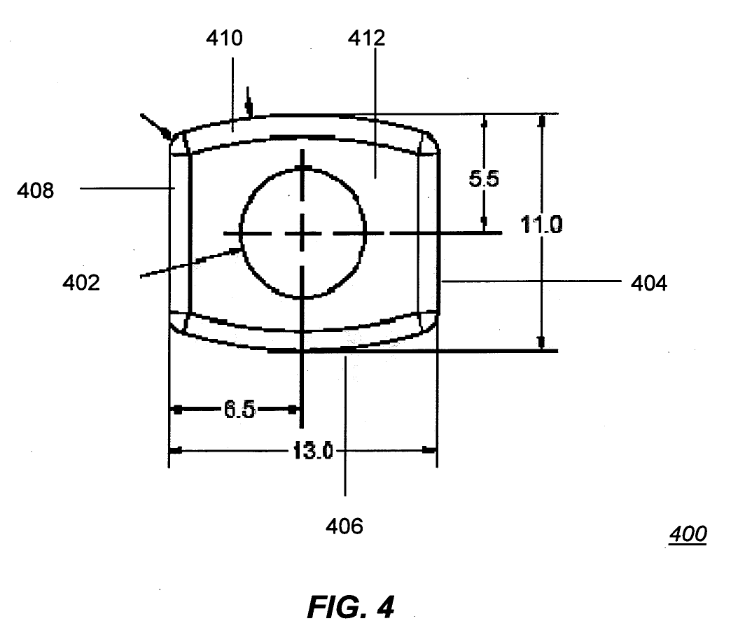

FIG. 3 is a three-dimensional top view of a first embodiment of a cervical intervertebral fusion device of the present invention. The cervical device 300 conforms in size and shape with the endplates of the adjacent vertebrae between which the cervical device 300 is to be implanted. The cervical device 300 is made of an allograft material which helps in the formation of new bone to fuse the two vertebral bodies together.

The bones can be from any source, including animals such as cows or pigs, e.g. xenograft bone. In one embodiment, the intervertebral fusion device is made of allogenic cortical bone received from animal long bones, such as portions of a femur bone or a contiguous femur bone. The cortical long bones are cut perpendicular to the bone axis and the marrow is removed to obtain annular shapes. The annular rings are then machined using appropriate equipment, known to persons of ordinary skill in the art, and are finally cleaned for implantation.

In one embodiment, the present invention is fabricated from one solid piece of cortical bone cored from a femur bone. The solid piece of bone is advantageous in that it does not require pins to be held together. In conventional implants, cortical bones are cut into a plurality of separate pieces that need to be secured to each other using pins derived from bone material. This typically results in breakage of the implant.

The resultant annular cervical device is, in one embodiment, lyophilized. The lyophilized process dries the cervical device 300 by freezing under high vacuum pressure. The cervical device 300 is enveloped in a lyophilized pouch for further protection. In one embodiment, the cervical device 300 is used as an implant deployed in an Anterior Cervical Fusion (ACF) procedure. In another embodiment, the cervical device 300 is used as an implant deployed in a Posterior Cervical Fusion (PCF) procedure.

In one embodiment, the cervical intervertebral fusion device 300 is substantially cuboidal in shape having four substantially planar, yet slightly curved sides 302, 304, 306, and 308. The implantable cervical fusion device further comprises a centrally disposed and cylindrical shaped hollow interior or through cavity 318, top surface 320 and bottom surface 322. In one embodiment, the implantable cervical fusion device is slightly tapered so that top surface 320 is slightly larger than bottom surface 322.

In one embodiment, top surface 320 and bottom surface 322 comprise smooth, flat, planar surfaces for maximum contact and fusion.

In another embodiment, the top and bottom surfaces comprise a macro-structure having plurality of footings and grooves that define a space, said space being covered by a micro-structure. In one embodiment, the space defined by the grooves and footings is at least 3 mm. In another embodiment, the footings comprise right triangles, have sharp ends, penetrate the endplates of the vertebrae, structurally degenerate to create an increased surface area for fusion, or have a minimum height of 0.5 mm.

In one embodiment, the grooves allow bone in-growth or are positioned at the front of each said footing.

In one embodiment, the micro-structure mimics cancellous bone architecture and/or has osteoconductive or osteoinductive qualities.

The micro-structure preferably has a roughness of the order of 100-250 micrometer, although other roughness ranges, such as 50 to 1000 microns, may be employed. This microstructure helps improve the initial stability of the intervertebral fusion device due to increased friction. The osteoconductive and/or oasteoinductive nature of the microtexture helps in the promotion of bone apposition. In one embodiment, the microtexture comprises the coating described in U.S. Pat. No. 4,206,516, which is incorporated herein by reference. In another embodiment, the microtexture comprises the coating described in U.S. Pat. No. 4,865,603, which is also incorporated herein by reference.

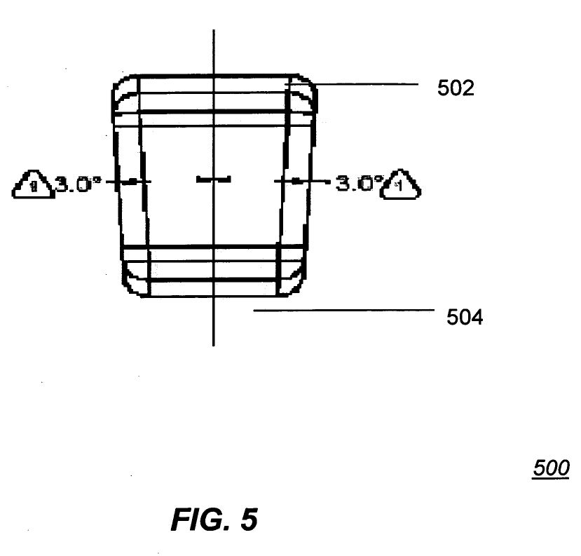

FIG. 4 is a two-dimensional top view of one embodiment of the cervical intervertebral fusion device of the present invention, shown in FIG. 3. In one embodiment of the present invention, the inner diameter of the through cavity 402 ranges from 5.0 mm to 6.2 mm. The diameter of the through cavity 402 is generally greater at the top surface 412 than the bottom surface, owing to the slightly tapered shape of cervical intervertebral fusion device 400.

The anterior and posterior diameters are concentric and aligned at the same center point with respect to each diameter. The end-to-end lengths (or diameters) between the center point of substantially parallel sides 404, 408 range from 11 mm to 13 mm and parallel sides 406, 410 have end-to-end lengths (or diameters) ranging from 13 mm to 15 mm. In one embodiment, parallel sides 404 and 408 have diameters of 11 mm. In one embodiment, parallel sides 406 and 410 have diameters of 13 mm. Thus, in one embodiment, the distance between the center of the through cavity 402 and each of parallel sides 406, 410 is 5.5 mm while the distance between the center of the through cavity 402 and each of parallel sides 404, 408 is 6.5 mm.

It should be noted herein that although exemplary ranges for the anterior and posterior diameters have been provided, the implantable cervical intervertebral fusion device of the present invention can be varied in size, depending upon factors such as, but not limited to individual patient size and location of implant.

In one embodiment of the present invention, the overall height of the cervical intervertebral fusion device 400 ranges from 5 mm to 10 mm, and can be varied in 1 mm increments. The variety of available heights of the cervical intervertebral fusion device provides the surgeons with a wide selection of appropriate cervical intervertebral fusion devices in accordance with individual patient vertebral column specifications.

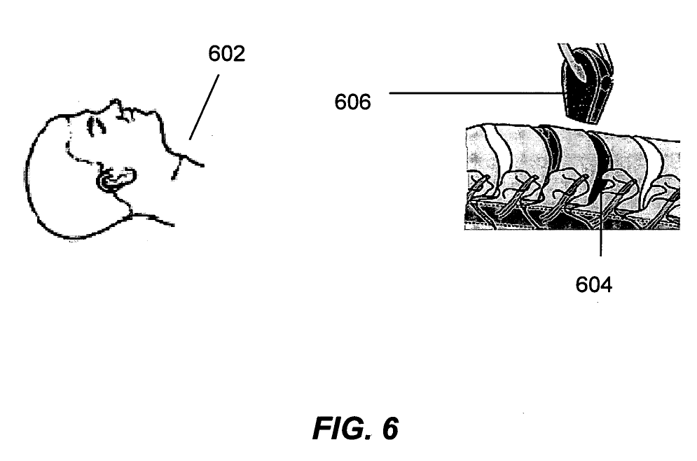

FIG. 5 is a two-dimensional side view of one embodiment of the cervical intervertebral fusion device of the present invention, shown in FIG. 3. As can be shown in FIG. 5, in one embodiment, the implantable cervical fusion device 500 is slightly tapered so that top surface or area 502 is slightly larger than bottom surface or area 504. In one embodiment, the cervical intervertebral fusion device 500 of the present invention forms an angle of three (3) degrees on each side of the device for maintaining the natural lordosis of the cervical spine.

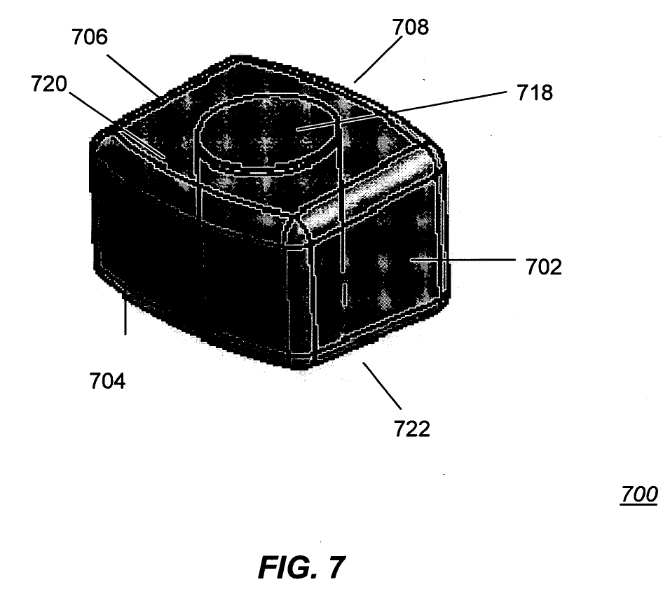

FIG. 6 is a schematic diagram of an anterior cervical fusion (ACF) surgery illustrating the implantation of the cervical intervertebral fusion device of the present invention, shown in FIG. 3. A patient is first stabilized for operation. The surgeon then makes a small incision 602 on the front side of a patient's neck, on one side. The location of the incision is carefully selected to expose disc space 604 which lies in between the vertebrae. A surgical drill is employed to enlarge the disc space. Cervical intervertebral fusion device 606 is then placed in disc space 604, where it fuses with the vertebrae. In one embodiment, small, specialized metal plates are also placed on the front of the cervical spine in order to increase the stability of the spine immediately after the operation.

Optionally, the cervical intervertebral fusion device further comprises a cancellous center which is slightly raised. In one embodiment, the cavity of the cervical device is filled with a precision-milled cancellous center which is slightly raised at both the top and bottom for enhancing fast and speedy fusion of the device. In one embodiment, the cancellous center is slightly raised on the order of 0.25 mm. Additionally, the cancellous center is continuous and a solid piece of cortical bone forms a cortical-cancellouous center. The cortical-cancellous center enables the cervical intervertebral fusion device to fuse with the adjacent vertebrae effectively without any pins thus making the entire process both economic and effective.

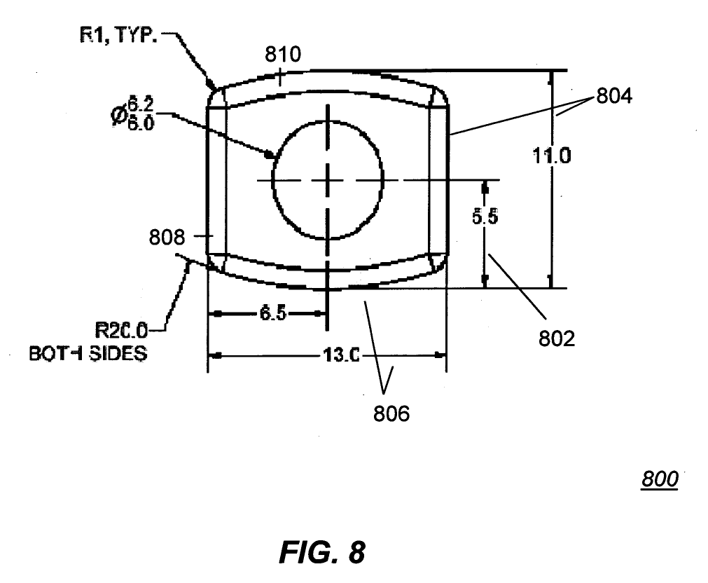

FIG. 7 is a three-dimensional top view of a second embodiment of a cervical intervertebral fusion device of the present invention, having a filled through cavity. The cervical device 700 conforms in size and shape with the endplates of the adjacent vertebrae between which the cervical device 700 is to be implanted. The cervical device 700 is made of an allograft material which helps in the formation of new bone to fuse the two vertebral bodies together.

The bones can be from any source, including animals such as cows or pigs, e.g. xenograft bone. In one embodiment, the intervertebral fusion device is made of allogenic cortical bone received from human long bones such as the femur bone. The cortical long bones are cut perpendicular to the bone axis and the marrow is removed to obtain annular shapes. The annular rings are then machined using appropriate equipment, known to persons of ordinary skill in the art, and are finally cleaned for implantation.

In one embodiment, the present invention is fabricated from one solid piece of cortical bone cored from a femur bone. The solid piece of bone is advantageous in that it does not require pins to be held together. In conventional implants, cortical bones are cut into a plurality of separate pieces that need to be secured to each other using pins derived from bone material. This typically results in breakage of the implant.

The resultant annular cervical device is, in one embodiment, lyophilized. The lyophilized process dries the cervical device 700 by freezing under high vacuum pressure. The cervical device 700 is enveloped in a lyophilized pouch for further protection. In one embodiment, the cervical device 700 is used as an implant in an Anterior Cervical Fusion (ACF) procedure. In another embodiment, the cervical device 700 is used as an implant in a Posterior Cervical Fusion (PCF) procedure.

In one embodiment, the cervical intervertebral fusion device 700 is substantially cuboidal in shape having four substantially planar, yet slightly curved sides 702, 704, 706, and 708. The implantable cervical fusion device further comprises a centrally disposed and cylindrical shaped hollow interior or through cavity 718, top surface 720 and bottom surface 722. In one embodiment, the implantable cervical fusion device is slightly tapered so that top surface 720 is slightly larger than bottom surface 722.

In one embodiment, top surface 720 and bottom surface 722 comprise smooth, flat, planar surfaces for maximum contact and fusion.

In one embodiment, the top and bottom surfaces comprise a macro-structure having plurality of footings and grooves that define a space, said space being covered by a micro-structure. In one embodiment, the space defined by the grooves and footings is at least 3 mm. In another embodiment, the footings comprise right triangles, have sharp ends, penetrate the endplates of the vertebrae, structurally degenerate to create an increased surface area for fusion, or have a minimum height of 0.5 mm.

In one embodiment, the grooves allow bone in-growth or are positioned at the front of each said footing.

In one embodiment, the micro-structure mimics cancellous bone architecture and/or has osteoconductive or osteoinductive qualities. The characteristics details of a preferred microstructure have already been described with respect to the first embodiment and FIG. 3 and will not be repeated herein.

In one embodiment of the present invention, the through cavity 718 is filled with a cancellous center. The cancellous center is precision milled for maximum graft host interface. In one embodiment, the cancellous center is slightly raised relative to the top surface.

In one embodiment, through cavity 718 is packed with porous osteoconductive or osteoinductive graft material to promote bony end to end growth and fusion. The use of osteoconductive or osteoinductive material has been described in detail above and will not be repeated herein.

In another embodiment, through cavity 718 further comprises osteogenic graft material, such as bone morphogenetic protein (BMP). BMP plays a key role in the transformation of mesenchymal cells into bone and cartilage.

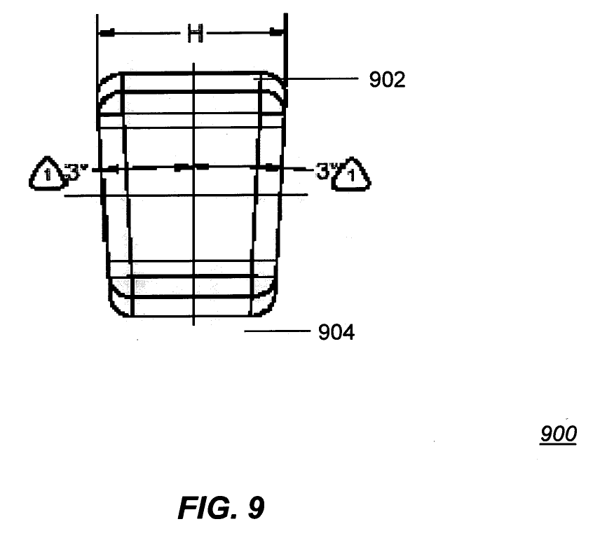

FIG. 8 is a two-dimensional top view of one embodiment of the cervical intervertebral fusion device of the present invention, shown in FIG. 7. In one embodiment of the present invention, the inner diameter of the through cavity 802 ranges from 5.0 mm to 6.2 mm. The diameter of the through cavity 802 is generally greater at the top surface 812 than the bottom surface, owing to the slightly tapered shape of cervical intervertebral fusion device 800.

The anterior and posterior diameters are concentric and aligned at the same center point with respect to each diameter. The end-to-end lengths (or diameters) between the center point of substantially parallel sides 804, 808 range from 11 mm to 13 mm and parallel sides 806, 810 have end-to-end lengths (or diameters) ranging from 13 mm to 15 mm. In one embodiment, parallel sides 804 and 808 have diameters of 11 mm. In one embodiment, parallel sides 806 and 810 have diameters of 13 mm. Thus, in one embodiment, the distance between the center of the through cavity 802 and each of parallel sides 806, 810 is 5.5 mm while the distance between the center of the through cavity 402 and each of parallel sides 804, 808 is 6.5 mm.

It should be noted herein that although exemplary ranges for the anterior and posterior diameters have been provided, the implantable cervical intervertebral fusion device of the present invention can be varied in size, depending upon factors such as, but not limited to individual patient size and location of implant.

In one embodiment of the present invention, the overall height of the cervical intervertebral fusion device 800 ranges from 5 mm to 10 mm, and can be varied in 1 mm increments. The variety of available heights of the cervical intervertebral fusion device provides the surgeons with a wide selection of appropriate cervical intervertebral fusion devices in accordance with individual patient vertebral column specifications.

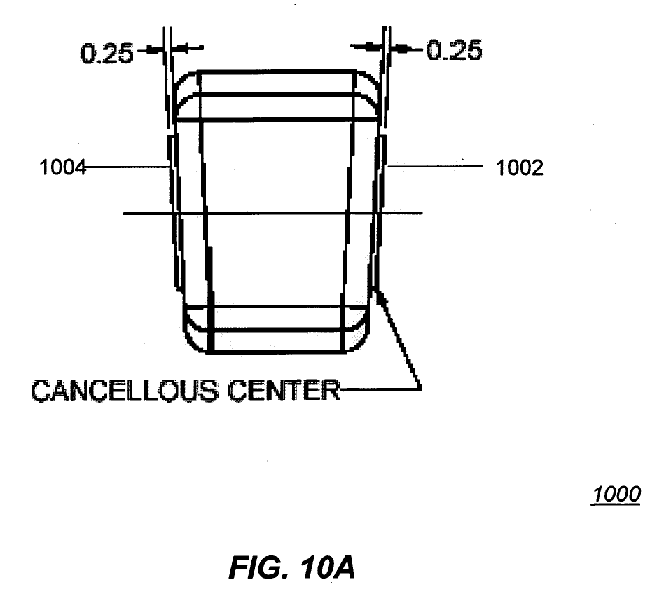

FIG. 9 is a two-dimensional side view of one embodiment of the cervical intervertebral fusion device of the present invention, shown in FIG. 7. As can be shown in FIG. 9, in one embodiment, the implantable cervical fusion device 900 is slightly tapered so that top surface or area 902 is slightly larger than bottom surface or area 904. In one optional embodiment, the cervical intervertebral fusion device 900 of the present invention forms an angle of three degrees on each side of the device for maintaining the natural lordosis of the cervical spine.

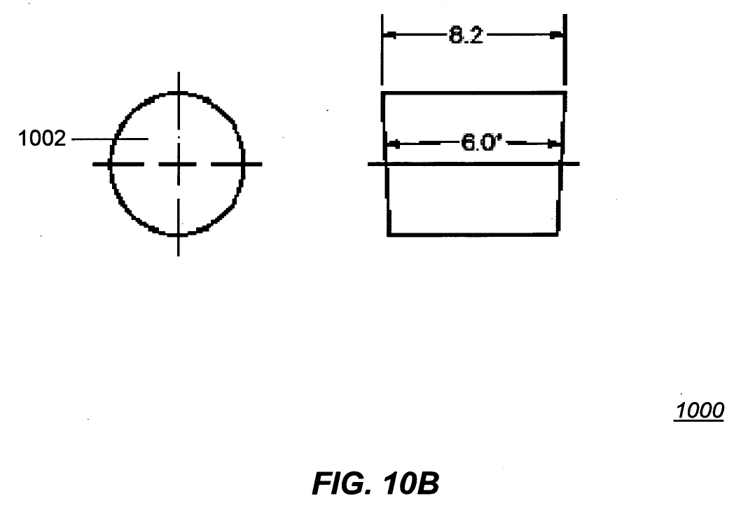

FIG. 10A is a two-dimensional side view of one embodiment of a cervical intervertebral fusion device 1000 of the present invention, having a cancellous center. In one embodiment, the cancellous center 1002, 1004 is located on each side of the cervical intervertebral fusion device above the cortical surface. In one embodiment, the amount the cancellous center is raised above the cortical surface ranges from 0.1 mm to 0.3 mm. In one embodiment, the cancellous center 1002, 1004 is raised 0.25 mm above the cortical surface.

FIG. 10B is a two-dimensional view of one embodiment of a cervical intervertebral fusion device of the present invention, having a cancellous center, also shown in FIG. 10A. In one embodiment, the through cavity 1002 of the device 1000 is filled with a precision-milled cancellous center ranging from 0.1 mm to 0.3 mm. In one embodiment, the through cavity 1002 is filled with 0.1 mm precision-milled cancellous center. The oversized cancellous center provides maximum surface contact between the vertebrae where it is implanted and helps in speedy fusion of the cervical device.

In one embodiment, the cervical intervertebral fusion device comprises a smooth surface without castling or rough texture.

In addition, in one embodiment, the device is radiused to enable ease of insertion without altering the basic structure of the vertebrae thus preserving the endplates.

Optionally, in order to maintain the natural lordosis of the cervical spine, an angle of three degrees on both sides of the cervical intervertebral fusion device is provided which results in an overall angle of six degrees. The three degree angle at both sides of cervical device enables appropriate sagittal alignment with respect to the cervical spine.

In one embodiment, an offset of 0.2 mm is provided to adjust the height and width of the cervical intervertebral fusion device for proper alignment with the cervical spines of individual patients.

In one optional embodiment, the intervertebral fusion device is coated with an osteoconductive surfacing or surface treatment, or any osteoinductive or other bone growth stimulation coating material to further promote the formation of new bone.

In addition, the terminals of the cervical intervertebral fusion device are sterilized, e.g. made free from live bacteria or other microorganisms so that it can be used directly while operating thus making the entire process considerably fast.

Optionally, the above-mentioned embodiments may be fabricated from PEEK or carbon fiber devices, or ceramics or any other suitable material.

The above examples are merely illustrative of the many applications of the system of present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention is not to be limited to the details given herein, but may be modified within the scope of the appended claims.

We claim:

1. An implantable intervertebral device, comprising: a single bone body substantially conforming in size and shape with the endplates of adjacent vertebrae wherein, the bone body comprises a top surface, a bottom surface and, a cavity wherein said cavity extends from said top surface to said bottom surface.

2. The device of claim 1, wherein the said cavity is filled with osteoconductive or osteoinductive graft material.

3. The device of claim 1, wherein the said cavity is filled with a high precision cancellous center.

4. The device of claim 3, wherein the cancellous center is raised relative to said single bone body.

5. The device of claim 4, wherein the cancellous center is raised in the range of 0.25 mm to 0.5 mm.

6. The device of claim 1, wherein the said bone body is cut and machined into annular shapes.

7. The device of claim 1, wherein the bottom surface is a flat planar surface.

8. The device of claim 1, wherein the bone body has lateral corners which are rounded or chamfered.

9. The device of claim 1, wherein the single bone body comprises a contiguous piece of femur bone.

10. The device of claim 1, wherein the said cavity is filled with bone morphogenetic protein.

11. An implantable intervertebral device, comprising: a single bone body made of a contiguous femur bone wherein said single bone body substantially conforms in size and shape with the endplates of adjacent vertebrae and wherein the single bone body comprises a top surface, a bottom surface and a cavity.

12. The device of claim 11 wherein said cavity extends from said top surface to said bottom surface and wherein said cavity is filled with osteoconductive or osteoinductive graft material.

13. The device of claim 11, wherein the said cavity is filled with bone morphogenetic protein.

14. The device of claim 11, wherein the said cavity is filled with a high precision cancellous center.

15. The device of claim 14, wherein the cancellous center is raised relative to said single bone body in the range of 0.25 mm to 0.5 mm.

16. The device of claim 11, wherein the said bone body is cut and machined into annular shapes.

17. The device of claim 11, wherein the bottom surface is a flat planar surface.

18. The device of claim 11, wherein the bone body has lateral corners which are rounded or chamfered.

19. An implantable intervertebral device, comprising: a single bone body made of a contiguous femur bone wherein said single bone body substantially conforms in size and shape with the endplates of adjacent vertebrae and wherein the single bone body comprises a top surface, a bottom surface and a cavity, wherein said cavity extends from said top surface to said bottom surface and wherein said cavity is filled with osteoconductive or osteoinductive graft material.

20. The device of claim 19, wherein the said cavity is filled with bone morphogenetic protein and is raised relative to said single bone body in the range of 0.25 mm to 0.5 mm.
